(19) United States
(12) Patent Application Publication
(10) Pub. No.: US 2004/0170992 A1
(43) Pub. Date: Sep. 2, 2004

(54) Diagnostic polymorphisms of tgf-beta1 promoter

(76) Inventor: David W. Moskowitz, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/467,149

(22) PCT Filed: Mar. 26, 2001
(86) PCT No.: PCT/US01/09743

Related U.S. Application Data
(60) Provisional application No. 60/191,922, filed on Mar. 24, 2000.

(51) Int. Cl. C12Q 1/68; C07H 21/04

(52) U.S. Cl. 435/6; 536/23.2

(57) ABSTRACT

Disclosed are single nucleotide polymorphisms (SNPs) associated with hypertension and end stage renal disease due to hypertension. Also disclosed are methods for using SNPs to determine susceptibility to end stage renal disease and hypertension; nucleotide sequences containing SNPs; kits for determining the presence of SNPs; and methods of treatment or prophylaxis based on the presence of SNPs.

### CROSS REFERENCE TO RELATED APPLICATIONS

This application claims the benefit of U.S. provisional application serial No. 60/191,922, filed Mar. 24, 2000, which is incorporated herein by reference in its entirety.

### BACKGROUND

This invention relates to detection of individuals at risk for pathological conditions based on the presence of single nucleotide polymorphisms (SNPs).

During the course of evolution, spontaneous mutations appear in the genomes of organisms. It has been estimated that variations in genomic DNA sequences are created continuously at a rate of about 100 new single base changes per individual (Kondrashow, J. Theor. Biol., 175:583-594, 1995; Crow, Exp. Clin. Immunogenet., 12:121-128, 1995). These changes, in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphism are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome are single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives (alleles) exist in a population. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions (e.g. A->G). Nucleotide substitutions are of two types. A transition is the replacement of one purine by another purine or one pyrimidine by another pyrimidine. A transversion is the replacement of a purine for a pyrimdine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998). The frequency of SNPs varies with the type and location of the change. In base substitutions, two-thirds of the substitutions involve the C<->T (G<->A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct, as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect, where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP) (U.S. Pat. Nos. 5,324,631; 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases have also been used to detect SNPS.

There is growing recognition that SNPs can provide a powerful tool for the detection of individuals whose genetic make-up alters their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

One disease for which the discovery of markers to detect increased genetic susceptibility is critically needed is end-stage renal disease. End-stage renal disease (ESRD) is defined as the condition when life becomes impossible without replacement of renal functions either by kidney dialysis or kidney transplantation. Hypertension (HTN) and non-insulin dependent diabetes (NIDDM) are the leading causes of end-stage renal disease (ESRD) nationally (United States Renal Data System, Table IV-3, p. 49, 1994). There is currently an epidemic of ESRD, due mainly to the aging of the American population. The ESRD epidemic is of special concern among African Americans where the incidence of ESRD is four- to six-fold higher than for Caucasians (Brancati et al., J. Am. Med. Assoc., 268:3079-3084, 1992), but where treatment of hypertension, a causative factor in ESRD, is less effective (Walker et al., J. Am. Med. Assoc., 268:3085-3091, 1992).

There are currently 200,000 patients with ESRD receiving renal replacement therapy (dialysis or renal transplantation), with an annual cost of $13 billion. These numbers will certainly increase as the population of the nation continues to age. Since 1980, when complete data became available for the first time, most new cases of ESRD have been ascribed to NIDDM or hypertension. The incidence of ESRD due to NIDDM or hypertension is still increasing, suggesting that the U.S. is in the early phase of an epidemic of ESRD. Preventing ESRD would save at least $30,000 per patient, per year in dialysis costs alone, as well as enhance the patient's quality of life and ability to work. It is clearly the ideal method of cost-containment for renal disease. Without effective prevention of ESRD, the nation will instead be forced to adopt less humane methods of cost-containment, such as denial of access (gate-keeping), or rely upon unrealistic expectations about patient reimbursement rates, etc.

Transforming growth factor beta (TGF-β1) is a multifunctional polypeptide growth factor implicated in a variety of renal diseases. Almost every cell in the body has been shown to make some form of TGF-β, and almost every cell has receptors for TGF-β, the context of which determines their functionality. The transforming growth factor-β system is also a likely mediator of renal apoptosis. TGF-β is intimately connected with glomerular sclerosis, mesangial matrix expansion, and tubulointerstitial fibrosis in experimental rodent models and human glomerulnephritis (Border et al., Kidney Intl., 47 (Suppl. 49):S-59-S-61, 1995). Of the three isoforms available, TGF-β1 has been implicated most consistently in pathologic fibrosis (Khalil et al., Am. J. Respir. Cell. Mol. Biol., 14:131-138, 1996). Numerous animal and human studies have already linked the progression of renal disease, especially its hallmark pathology of interstitial fibrosis and glomerular sclerosis, to increased signaling by TGF-β1. (August P, et al. Curr. Hypertens. Rep., 2:184-91, 2000). Clouthier, et al. demonstrated in 1997 that overexpression of TGF-β1 in rat kidneys resulted in fibrosis and glomerular disease, eventually leading to complete loss of renal function (Clouthier, et al., J. Clin. Invest., Dec. 1;100:2697-713 (1997)).

Signaling by TGF-β1 involves specific binding of the ligand to the type II TGF-β1 receptor (abbreviated as TGFβ-RII), present on the plasma membrane of target cells such as fibroblasts in the case of glomerular and intersititial fibrosis. This receptor-ligand complex then heterodimerizes with the type I TGF-β1 receptor (abbreviated as TGFβ-RI). TGFβ-RI is constitutively active. Like the concentrations of ligand (TGF-β1) and TGFβ-RI, the concentration of TGF β-R11 in the plasma membrane is likely to be rate-limiting for signaling by TGF-β1. All elements of the pathway appear to be subject to complex regulation. TGF-β1 signaling has been identified, and methods of developing therapies based on these regulatory reactions have been characterized (for example, see Souchelnytokyi, et al., U.S. Pat. No. 6,103,869, or Falb, U.S. Pat. No. 6,099,823).

Activation of protein kinase C early during compensatory renal growth (CRF) would have the effect of stimulating TGF-β1 production, since the TGF-β1 promoter contains AP-1 sites (Kim et al., J. Biol. Chem., 264:402-408, 1989). Angiotensin II has been shown to induce TGF-β1 expression in renal mesangial cells, endothelial cells, and proximal tubular epithelial cells. Thus, greater induction of TGF-β1, or greater expression of its two main receptors (TGFβ-RI and TGFβ-RII), may occur in patients who progress to ESRD compared to patients who never develop CRF. Unlike the case with renal failure, TGF-β1 signaling has not been implicated in essential hypertension yet.

If the level of TGFβ-RII gene product (i.e. protein) is proportional to the level of mRNA, and the mRNA level is proportional to the transcriptional rate of the gene, then a SNP which disrupts a transcriptional activator site would be expected to decrease both the rate of transcription of the gene and the eventual concentration of TGFβ-RII in the plasma membrane of cells which express this protein. The net effect of such a SNP is expected to be protection against renal failure.

Since the coding sequence of TGF-β1 is identical between mouse and human, a period of evolutionary divergence of greater than 100 hundred million years, no human polymorphisms in the coding sequence are expected. Thus the TGF-β1 promoter and introns would be more likely candidates for genetic variants than the exons of the TGF-β1 structural gene. The promoter sequences and the structural genes for TGFβ-RI and TGFβ-RII are also likely candidates for genetic variations.

Those of ordinary skill in the art will recognize that alterations in the regulatory region of a gene, i.e. promoter, can produce substantive changes in the timing and quantity of the production of said gene's product. GC box elements are a relatively common regulatory motif (2.12 matches/1000 bases of random genomic DNA in vertebrates). Mutations in a GC box located at −90 of the human β-globin transcription startpoint result in suppression of transcription to as low as 10% of the normal level (Lewin, B. Genes VII; New York: Oxford University Press, 1999; pp. 634-635). If the level of TGFβ-RII gene product (i.e. protein) is proportional to the level of mRNA, and the mRNA level is proportional to the transcriptional rate of the gene, then a SNP which disrupts a transcriptional activator site would be expected to decrease both the rate of transcription of the gene and the eventual concentration of TGFβ-RII in the plasma membrane of cells which express this protein. The net effect of such a SNP is expected to be protection against renal failure.

An ideal approach to prevention of ESRD would be the identification of any genes that predispose an individual to ESRD early enough to be able to counteract this predisposition. Knowledge of ESRD-predisposing genes is essential for truly effective delay, or, ideally, prevention of ESRD.

### SUMMARY

The present inventor has discovered novel single nucleotide polymorphisms (SNPs) associated with the development of hypertension and/or end-stage renal disease in patients with hypertension. As such, these polymorphisms provide a method for diagnosing a genetic predisposition for the development of hypertension or end-stage renal disease in individuals. Information obtained from the detection of SNPs associated with the development of these diseases is of great value in the treatment and prevention of the diseases.

Accordingly, one aspect of the present invention provides a method for diagnosing a genetic predisposition for hypertension and/or end-stage renal disease in a subject, comprising obtaining a sample containing at least one polynucleotide from the subject, and analyzing at least the polynucleotide to detect a genetic polymorphism wherein said genetic polymorphism is associated with an altered susceptibility to developing hypertension and/or end stage renal disease.

Another aspect of the present invention provides an isolated nucleic acid sequence comprising at least 10 contiguous nucleotides from SEQ ID NO: 1, or their complements, wherein the sequence contains at least one polymorphic site associated with a disease and in particular hypertension and/or end-stage renal disease.

Yet another aspect of the invention is a kit for the detection of a polymorphism comprising, at a minimum, at least one polynucleotide of at least 10 contiguous nucleotides of SEQ ID NO: 1, or their complements, wherein the at least one polynucleotide contains at least one polymorphic site associated with hypertension and/or end-stage renal disease.

Yet another aspect of the invention provides a method for treating hypertension and/or end stage renal disease comprising, obtaining a sample of biological material containing at least one polynucleotide from the subject; analyzing the polynucleotide to detect the presence of at least one polymorphism associated with these diseases; and treating the subject in such a way as to counteract the effect of any such polymorphism detected.

Still another aspect of the invention provides a method for the prophylactic treatment of a subject with a genetic predisposition to hypertension and/or end stage renal disease comprising, obtaining a sample of biological material containing at least one polynucleotide from the subject; analyzing the polynucleotide to detect the presence of at least one polymorphism associated with these diseases; and treating the subject.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### DEFINITIONS

nt nucleotide

bp=base pair

kb=kilobase; 1000 base pairs

ESRD=end-stage renal disease

HTN=hypertension

NIDDM=noninsulin-dependent diabetes mellitus

CRF=chronic renal failure

T-GF=tubulo-glomerular feedback

CRG=compensatory renal growth

MODY=maturity-onset diabetes of the young

RFLP=restriction fragment length polymorphism

MASDA=multiplexed allele-specific diagnostic assay

MADGE=microtiter array diagonal gel electrophoresis

OLA=oligonucleotide ligation assay

DOL=dye-labeled oligonucleotide ligation assay

SNP=single nucleotide polymorphism

PCR=polymerase chain reaction

“polynucleotide” and “oligonucleotide” are used interchangeably and mean a linear polymer of at least 2 nucleotides joined together by phosphodiester bonds and may consist of either ribonucleotides or deoxyribonucleotides.

“sequence” means the linear order in which monomers occur in a polymer, for example, the order of amino acids in a polypeptide or the order of nucleotides in a polynucleotide.

“polymorphism” refers to a set of genetic variants at a particular genetic locus among individuals in a population.

“promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns”) between individual coding segments (“exons”). A promoter is herein considered as a part of the corresponding gene. Coding refers to the representation of amino acids, start and stop signals in a three base “triplet” code. Promoters are often upstream (“5“to”) the transcription initiation site of the gene.

“gene therapy” means the introduction of a functional gene or genes from some source by any suitable method into a living cell to correct for a genetic defect.

“wild type allele” means the most frequently encountered allele of a given nucleotide sequence of an organism.

“genetic variant” or “variant” means a specific genetic variant which is present at a particular genetic locus in at least one individual in a population and that differs from the wild type.

As used herein the terms “patient” and “subject” are not limited to human beings, but are intended to include all vertebrate animals in addition to human beings.

As used herein the terms “genetic predisposition”, genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (wild type) by at least 1%, preferably by at least 2%, more preferably by at least 4% and more preferably still by at least 8%.

As used herein “isolated nucleic acid” means a species of the invention that is the predominate species present (e.g., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

Novel Polymorphisms

The present application provides four single nucleotide polymorphisms (SNPs) in the TGF-β1 promoter gene associated with and/or hypertension. The location of these SNPs associated with hypertension as well as the wild type and variant nucleotides are summarized in Table 13. The location of these SNPs associated with end stage renal disease due to hypertension as well as the wild type and variant nucleotides are summarized in Table 14.

Role of SNP-Typing

Because the complexity of transcription allows for factors of multiple functions to recognize the same regulatory elements, and the functional nature of TGF βsignaling is context-dependent, it is extraordinarily difficult to predict at this time the precise impact that natural genetic variation in these regions may have on human pathology. Therefore, the most immediate way to understand and benefit from the knowledge of this natural human variation is statistical analysis of diseased populations. Many statistical techniques exist for quantifying the association between disease genes and disease phenotypes; the most robust for dissecting complex diseases, e.g. end-stage renal disease, is the case-control study design (Risch, N. & Merikangas, K. Science 273, 1516-1517 (1996).)

The promoter region of the TGFβ-1 gene has been well characterized, and several polymorphisms, including the one disclosed below, have been screened for functional and pathological effects. Grainger, et al. found that allelic variants in the promoter are correlated with the circulating plasma concentration of TGFβ-1 protein (Grainger, et al., Hum. Mol. Genet., 8 (1): 93-97 (1999)). Other studies have found associations between TGFβ-1 promoter SNPs and cardiovascular disease (Cambien, et al. Hypertension 28, 881-887 (1996)).

Further, well-known genotyping techniques can be performed to type polymorphisms that are in close proximity to mutations in the target gene itself, including mutations associated with fibroproliferative, oncogenic or cardiovascular disorders. Such polymorphisms can be used to identify individuals of a population likely to carry mutations in the target gene e.g., TGF P type II receptor or a related gene. If a polymorphism exhibits linkage disequilibrium with mutations in the target gene e.g., TGF β type II receptor, the polymorphism can also be used to identify individuals in the general population who are likely to carry such mutations.

For example, Drazen et al. (U.S. Pat. No. 6,090,547) describe a technique using SSCP to detect substitution polymorphisms, and SSLP to detect insertion/deletion polymorphisms, in the coding and regulatory regions of the 5-lipoxygenase gene. Furthermore, they demonstrate that these polymorphisms can be usefully associated with asthmatic phenotypes, the knowledge of which is used to predict a response to conventional asthma therapy.

Also, Weber (U.S. Pat. No. 5,075,217) describes a DNA marker based on length (i.e. insertion/deletion) polymorphisms in blocks of (dC-dA)ₙ-(dG-dT)ₙ short tandem repeats. The average separation of (dC-dA)ₙ-(dG-dT)ₙ blocks is estimated to be 30,000-60,000 bp. Markers that are so closely spaced exhibit a high frequency co-inheritance, and are extremely useful in the identification of genetic mutations, such as, for example, mutations within TGFβ-RII or a related gene, and the diagnosis of diseases and disorders related to mutations in the target gene.

Also, Caskey et al. (U.S. Pat. No. 5,364,759) describe a DNA profiling assay for detecting short tri and tetra nucleotide repeat sequences. The process includes extracting the DNA of interest, such as the target gene, e.g., TGFβ-RII or a related gene, amplifing the extracted DNA, and labeling the repeat sequences to form a genotypic map of the individual's DNA.

For a further example of the use of genetic markers in disease diagnosis, see Shor, et al. U.S. Pat. No. 5,424,187.

Preparation of Samples

The presence of genetic variants in the above genes or their control regions, or in any other genes that may affect susceptibility to ESRD is determined by screening nucleic acid sequences from a population of individuals for such variants. The population is preferably comprised of some individuals with ESRD, so that any genetic variants that are found can be correlated with ESRD. The population is also preferably comprised of some individuals that have known risk for ESRD, such as individuals with hypertension, NIDDM, or chronic renal failure. The population should preferably be large enough to have a reasonable chance of finding individuals with the sought-after genetic variant. As the size of the population increases, the ability to find significant correlations between a particular genetic variant and susceptibility to ESRD also increases. Preferably, the population should have 10 or more individuals.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factor for the disease. The populations should preferably be large enough to have a reasonable chance to find correlations between a particular genetic variant and susceptibility to the disease of interest. In one embodiment, the population should have at least 10 individuals. In one embodiment, the population is preferably comprised of individuals who have known risk factors for ESRD such as individuals with hypertension, NIDDM, or CRF. In addition, the allele frequency of the genetic variant in a population or subpopulation with the disease or pathology should vary from its allele frequency in the population without the disease or pathology (control population) or the control sequence (wild type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorplism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

In one embodiment, direct sequencing is accomplished by pyrosequencing. In pyrosequencing, a sequencing primer is hybridized with a DNA template and incubated with the enzymes DNA polymerase, ATP sulfurylase, luciferase and apyrase, and the substrates, adenosine 5′ phosphosulfate (APS) and luciferin. The first of four deoxynucleotide triphosphates (DNTP) is added to the reaction and incorporated into the DNA primer strand if it is complementary to the base in the template. Each dNTP incorporation is accompanied by release of pyrophosphate (PPi) in an quantity equimolar to the amount of incorporated nucleotide. ATP sylfurylase then quantitatively converts the PPi to ATP in the presence of adenosine 5′ phosphosulfate. The ATP produced drives the luciferase mediated conversion of luciferin to oxyluciferin which generates visible light in amounts proportional to the amount of ATP. The amount of light produced is measured and is proportional to the number of nucleotides incorporated. The reaction is then repeated for each of the remaining dNTPs. For dATP, alfa-thio triphosphate (dATPS) is used since it is efficiently utilized by DNA polymerase but not by luciferase. Methods for using pyrosequencing to detect SNPs are known in the art and can be found for example, in Alderbom et al., Genome Res. 10:1249-1258, 2000; Ahmadian et al., Anal. Biochem. 10: 103-110, 2000; and Nordstrom et al., Biotechnol. Appl. Biochem. 31:107-112, 2000.

RFLP analysis (see, e.g. U.S. Pat. No. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in<220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e. low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al., Trends Genet. 7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine (T) when mismatched with cytosine, guanine or thymine and the chemical reactivity of cytosine (C) when mismatched with thymine, adenine or cytosine (Cotton et al., Proc. Natl. Acad. Sci. USA, 85:4397-4401, 1988). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998 and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASOs that hybridize to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end, such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE) (Day and Humphries, Anal. Biochem., 222:389-395, 1994). In this assay restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sonmer et al., Mayo Cliii. Proc., 64:1361-1372 1989; Sarker et al., Anal. Biochem. 1990), allele-specific amplification (ASA), allele-specific PCR, and amplification refractory mutation system (ARMS) (Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determined whether an individual is homozygous or heterozygous for the mutation (Dutton and Sommer, BioTechniques,11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers has a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can be used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:346-354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled DNTP and noncompeting ddNTPs. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease and in particular, end-stage renal disease and hypertension. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are not suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an altered susceptability to a disease, condition or disorder, and in particular end-stage renal disease or hypertension. In one embodiment, the genetic variant is located at one of the polymorphic sites contained in Table 13 or 14. In another embodiment, the genetic variant is one of the variants contained in Table 13 or 14 or the complement of any of the variants contained in Table 13 or 14. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1, or the complement of SEQ ID NO: 1. In one preferred embodiment, the sequence contains at least one polymorphic site associated with a disease, and in particular end-stage renal disease or hypertension. In one embodiment, the polymorphic site is selected from the groups contained in Table 13 or 14. In another embodiment, the polymorphic site contains a genetic variant, and in particular, the genetic variants contained in Table 13 or 14 or the complements of the variants in Table 13 or 14. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in particular end-stage renal disease and hypertension. The kits contain, at a minimum, at least one polynucleotide of at least 10 contiguous nucleotides of SEQ ID NO 1, or the complement of SEQ ID NO: 1. In one embodiment, the polynucleotide contains at least one polymorphic site, preferably a polymorphic site selected from the groups contained in Table 13 or 14. Alternatively the 3′ end of the polynucleotide is immediately 5′ to a polymorphic site, preferably a polymorphic site contained in Table 13 or 14. In one embodiment, the polymorphic site contains a genetic variant, preferably a genetic variant selected from the groups contained in Table 13 or 14. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder and in particular end stage renal disease and hypertension caused either directly or indirectly by the presence of one or more single nucleotide polymorphisms. In this method, genetic material from a patient, for example, DNA, cDNA, RNA or mRNA is screened for the presence of one or more SNPs associated with the disease of interest. Depending on the type and location of the SNP, a treatment regime is designed to counteract the effect of the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder, and in particular end stage renal disease and hypertension due to the presence of one or more single nucleotide polymorphisms. In this embodiment, genetic material, such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

### EXAMPLES

Position of the single nucleotide polymorphism (SNP) is given according to the numbering scheme in GenBank Accession Number J04431. Thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://www.ncbi.ntm.nih.gov/

In the following examples, SNPs are written as “reference sequence nucleotide”→“variant nucleotide.” Changes in nucleotide sequences are indicated in bold print. The standard nucleotide abbreviations are used in which A=adenine, C=cytosine, G=guanine, T=thymine, M=A or C, R=A or G, W=A or T, S=C or G, Y=C or T, K=G or T, V=A or C or G, H=A or C or T; D=A or G or T; B=C or G or T; N=A or C or G or T.

### Example 1

### Detection of Novel Polymorphisms by Direct Sequencing of Leukocyte Genomic DNA

Leukocytes were obtained from human whole blood collected with EDTA. Control groups were normotensive individuals with healthy renal function. The hypertensive group consisted of patients with essential hypertension, but without evidence of renal disease (<2+proteinuria on random urinalysis: serum creatine less than or equal to 1.5 mg/dl). Blood was obtained from a group of 20 Caucasian males with ESRD due to hypertension, 23 Caucasian males with hypertension, and a control group of 29 Caucasian males. For the G562->A polymorphism, leukocytes were obtained from whole blood collected from African American men and women.

Genomic DNA was purified from the collected leukocytes using standard protocols well known to those of ordinary skill in the art of molecular biology (Ausubel et al., Short Protocol in Molecular Biology, 3^{rd} ed., John Wiley and Sons, 1995; Sambrook et al., Molecular Cloning, Cold Spring Harbor Laboratory Press, 1989; and Davis et al., Basic Methods in Molecular Biology, Elsevier Science Publishing, 1986). One hundred nanograms of purified genomic DNA was used in each PCR reaction.

Standard PCR reaction conditions were used. Methods for conducting PCR are well known in the art and can be found, for example, in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990. Specific primers used are given in the following examples.

PCR reactions were carried out in a total volume of 50 μl containing 10-15 ng leukocyte genomic DNA, 10 pmol of each primer, 200 nM deoxynucleotide triphosphates (dNTPs), 1.25 U Taq polymerase (Qiagen), 1× Qiagen PCR buffer (50 mM KCl, 10 mM Tris-HCl, pH 8.3, 1.5 mM MgCl₂, and 1×“Q” solution (Qiagen). After an initial 3 minutes denaturation at 94° C., 35 cycles were performed consisting of 1 minute denaturation at 94° C., 1 minute hybridization at 55° C., 2 minute extension at 72° C., followed by a final extension step of 5 minutes at 72° C., and 1 minute cooling at 35° C.

For the G563->A polymorphism, the PCR reactions were carried out as described above except as follows: after an initial 5 minutes denaturation at 94° C., 35 cycles were performed consisting of 45 seconds denaturation at 94° C., 45 second hybridization at 65° C., 45 second extension at 72° C., followed by a final extension step of 10 minutes at 72° C.

Post-PCR clean-up for all samples was performed as follows. PCR reactions were cleaned to remove unwanted primer and other impurities such as salts, enzymes, and unincorporated nucleotides that could inhibit sequencing. One of the following clean-up kits was used: Qiaquick-96 PCR Purification Kit (Qiagen) or Multiscreen-PCR Plates (Millipore, discussed below).

When using the Qiaquick protocol, PCR samples were added to the 96-well Qiaquick silica-gel membrane plate and a chaotropic salt, supplied as “PB Buffer,” was then added to each well. The PB Buffer causes DNA to bind to the membrane. The plate was put onto the Qiagen vacuum manifold and vacuum was applied to the plate in order to pull sample and PB Buffer through the membrane. The filtrate was discarded. Next, the samples were washed twice using “PE Buffer.” Vacuum pressure was applied between each step to remove the buffer. Filtrate was similarly discarded after each wash. After the last PE Buffer wash, maximum vacuum pressure was applied to the membrane plate to generate maximum airflow through the membrane in order to evaporate residual ethanol left from the PE Buffer. The clean PCR product was then eluted from the filter using “EB Buffer.” The filtrate contained the cleaned PCR product and was collected. All buffers were supplied as part of the Qiaquick-96 PCR Purification Kit. The vacuum manifold was also purchased from Qiagen for exclusive use with the Qiaquick-96 Purification Kit.

When using the Millipore Multiscreen-PCR Plates, PCR samples were loaded into the wells of the Multiscreen-PCR Plate and the plate was then placed on a Millipore vacuum manifold. Vacuum pressure was applied for 10 minutes, and the filtrate was discarded. The plate was then removed from the vacuum manifold and 100 μl of Milli-Q water was added to each well to rehydrate the DNA samples. After shaking on a plate shaker for 5 minutes, the plate was replaced on the manifold and vacuum pressure was applied for 5 minutes. The filtrate was again discarded. The plate was removed and 60 μl Milli-Q water was added to each well to again rehydrate the DNA samples. After shaking on a plate shaker for 10 minutes, the 60 μl of cleaned PCR product was transferred from the Multiscreen-PCR plate to another 96-well plate by pipetting. The Millipore vacuum manifold was purchased from Millipore for exclusive use with the Multiscreen-PCR plates.

Cycle sequencing was performed on the clean PCR product using an ABI Prism Big Dye Terminator Cycle Sequencing Ready Reaction kit (Perkin-Elmer). For a total volume of 20 μl, the following reagents were added to each well of a 96-well plate: 2.0 μl Terminator Ready Reaction mix, 3.0 μl 5× Sequencing Buffer (ABI), 5-10 μl template (30-90 ng double stranded DNA), 3.2 μM primer (primer used was the forward primer from the PCR reaction), and Milli-Q water to 20 μl total volume. The reaction plate was placed into a Hybaid thermal cycler block and programmed as follows: X 1 cycle: 1 degree/sec thermal ramp to 94° C., 94° C. for 1 min; X 35 cycles: 1 degree/sec thermal ramp to 94° C., then 94° C. for 10 sec, followed by 1 degree/sec thermal ramp to 50° C., then 50° C. for 10 sec, followed by 1 degree/sec thermal ramp to 60° C., then 60° C. for 4 minutes.

The cycle sequencing reaction product was cleaned up to remove the unincorporated dye-labeled terminators that can obscure data at the beginning of the sequence. A precipitation protocol was used. To each sequencing reaction in the 96-well plate 20 μl of Milli-Q water and 60 μl of 100% isopropanol was added. The plate was left at room temperature for at least 20 minutes to precipitate the extension products. The plate was spun in a plate centrifuge (Jouan) at 3,000×g for 30 minutes.

Without disturbing the pellet, the supernatant was discarded by inverting the plate onto several paper tissues (Kimwipes) folded to the size of the plate. The inverted plate, with Kimwipes in place, was placed into the centrifuge (Jouan) and spun at 700×g for 1 minute. The Kimwipes were discarded and the samples were loaded onto a sequencing gel.

Approximately 1 μl of sequencing product was loaded into each well of a 96-lane 5% Long Ranger (FMC single pack) gel. The running buffer consisted of 1×TBE (Tris Borate EDTA). The glass plates consisted of ABI 48-cm plates for use with a 96-lane 0.4 mm Mylar shark-tooth comb. A semi-automated ABI Prism 377-96 DNA sequencer was used (ABI 377 with 96-lane, Big Dye upgrades). Sequencing run settings were as follows: run module 48E-1200, 8 hr collection time, 2400 V electrophoresis voltage, 50 mA elecrophoresis current, 200 W electrophoresis power, CCD offset of 0, gel temperature of 51° C., 40 mW laser power, and CCD gain of 2.

The SEQUENCHER program (Gene Codes Corp., Ann Arbor, Mich.) was used to ensure that only a high-quality sequence was used for allele assignment. The 5′ end of the sequence was trimmed to a maximum of 25%, until there were fewer than 3 ambiguities. The 3′ end was defined as beginning 100 bases after the trimmed 5′ end. The 3′ end was similarly trimmed to remove any sequence containing 3 or more ambiguities in 25 nucleotides. If any ambiguous bases still remained at the 5′ or 3′ end, they were also removed. These settings are considerably stricter than the baseline default settings of the program. Individual sequences were excluded if they revealed less than 85% identity to the reference sequence (“dirty data algorithm,” SEQLENCHER program).

Prediction of potential transcription binding factor sites was performed using a commercially available software program [GENOMATIX MatInspector Professional; URL: http://genomatix.gsf.de/cgi-bin/matinspector/matinspector.pl; Quandt et al., Nucleic Acids Res., 23: 4878-4884 (1995)].

### Example 2

TABLE 1
G to T Substitution at Position 474 of Human TGF-β1 Promoter
ALLELE FREQUENCIES

| | G | T |
|---|---|---|
| CONTROL (n = 56 chromosomes): | 48 | 8 |
| Caucasian men | 86% | 14% |
| DISEASE | | |
| HYPERTENSION (n = 64 chromosomes): | 48 | 16 |
| Caucasian men | 75% | 25% |
| ESRD due to HTN (n = 34 chromosomes): | 27 | 7 |
| Caucasian men | 79% | 21% |

TABLE 2
GENOTYPE FREQUENCIES

| | G/G | G/T | T/T |
|---|---|---|---|
| CONTROL (n = 28 individuals): | 20 | 8 | 0 |
| Caucasian men | 71% | 29% | 0% |
| DISEASE | | | |
| HYPERTENSION (n = 32 individuals): | 16 | 16 | 0 |
| Caucasian men | 50% | 50% | 0% |
| ESRD due to HTN (n = 17 individuals): | 10 | 7 | 0 |
| Caucasian men | 59% | 41% | 0% |

PCR and sequencing were conducted as in Example 1. The sense primer was 5′-TGCATGGGGACACCATCTACAG-3′ (SEQ ID NO: 2) and the antisense primer was 5′-TCTTGACCACTGTGCCATCCTC-3′ (SEQ ID NO: 3). The 202 nucleotide PCR product spanned positions 421 to 622 of the human TGF-β1 gene (SEQ ID NO: 1).

As shown above, the frequency of the SNP (T allele) is higher (25% vs. 14%) in Caucasian male hypertensive patients than in control individuals. The frequency of the T allele is essentially the same for Caucasian male patients with ESRD due to hypertension as for white men with hypertension (21% vs. 25%). The genotype frequencies for the two disease categories are similar, and distinct from controls. The frequency of the G/T genotype increases from control patients (29%) to hypertensive white male patients (50%): the frequency of the G/T genotype in white men with ESRD due to hypertension (41%) is similar to the G/T genotype frequency in hypertensive white men (50%). These data suggest that the SNP “T” allele contributes towards hypertension.

The control sample approximates Hardy-Weinberg equilibrium, as expected. Hardy-Weinberg equilibrium is a term used to describe the distribution of genotypes at a biallelic locus in a stable population without recent genetic admixture, drift, or selection pressure. The equilibrium distribution is a binomial expansion of the two allele frequencies, p and q=1−p, i.e. (p+q)²=p²+2pq+q²=1.

A frequency of 0.86 for the G allele (“p”) and 0.14 for the T allele (“q”) among control individuals predicts genotype frequencies of 74% GIG, 24% G/T, and 2% T/T at Hardy-Weinberg equilibrium (p²+2pq+q²=1). The observed genotype frequencies were 71% G/G, 29% G/T, and 0% T/T, in close agreement with those predicted for Hardy-Weinberg equilibrium. The two disease categories diverge from Hardy-Weinberg equilibrium, which is consistent with this locus being disease-associated.

The G474-->T SNP is predicted to disrupt the following transcriptional regulatory sites in the TGF-β1 gene promoter:

a. The G to T substitution at position 474 results in disruption of a potential E47_{—}01 (E47) binding site whose 3′ terminus ends at nucleotide 464 on the (−) strand. The binding site consists of the complementary sequence to 5′-NNGNMCACCTGCNSN-3′. This SNP replaces the indicated G with a T. E47_{—}01 binding sites occur rather rarely at 0.11 matches per 1000 base pairs of random genomic sequence in vertebrates, suggesting that the presence of this E-box in the TGF-β1 promoter is meaningful.

E47 is a basic helix-loop-helix (bHLH) protein which is ubiquitously expressed in tissues. It can form either homodimers, or heterodimers with another group of tissue-specific (so-called Class II) bHLH proteins, such as MyoD (see below).

The effect of disrupting the E47 binding site in the TGF-β 1 promoter is unknown and difficult to predict. E47 homodimers stimulate transcription of some genes, such as the immunoglobulin heavy chain and insulin. However, overexpression of E47 inhibits transcription of the glucagon gene through an E47/BETA2 heterodimer (Dumonteil, et al., J. Biol. Chem. 273:19945-19954, 1998).

That E47 may activate the TGF-β1 gene is suggested by the observation that E47 induces growth arrest of fibroblasts at the G1-S transition in the cell cycle (Peverali et al., EMBO J. 13:4291-4301, 1994). Inhibition of cell proliferation is consistent with increased signaling by TGF-β1.

If E47 is a transcriptional activator, disruption of its binding site in the TGF-β1 promoter is expected to result in a lower rate of TGF-β1 signaling. There is as yet no known association of TGF-β1 with essential hypertension. Association of the G474-->T SNP with essential hypertension suggests a novel mechanism for this disease.

b. The G to T substitution disrupts a potential E47_{—}02 binding site whose 3′ terminus ends at nucleotide 464 on the (−) strand. The binding site consists of the complementary sequence to 5′-NNKAACACCTGYKNNN-3′ (SEQ ID NO: 4); this SNP replaces the indicated G with a T. E47_{—}02 binding sites occur relatively rarely with a frequency of 0.27 times per 1000 base pairs of random genomic sequence in vertebrates. The significance of the disruption of the E47_{—}02 binding site is thought to be the same as for the E47_{—}01 site discussed above.

c. The G to T substitution disrupts a potential LMO2COM (complex of Lmo2 bound to Tal-1 and E2A protein [E47]) binding site whose 3′ terminus ends at nucleotide 466 on the (−) strand. The binding site consists of the complementary sequence to 5′-NNNCACCTGCNNS-3′ (SEQ ID NO: 5). This SNP replaces the indicated G with a T. LMO2COM binding sites occur rather frequently at 1.11 matches per 1000 base pairs of random genomic sequence in vertebrates. The effect of disrupting the Lmo2 complex binding site in the TGF-β 1 promoter is unknown and difficult to predict.

d. There is disruption of a potential MyoD_Q6 (myoblast determining factor) binding site whose 3′ terminus ends at nucleotide 467 on the (−) strand. The binding site consists of the complementary sequence to 5′-RNCAGNTGNN-3′ (SEQ ID NO: 6). This SNP replaces the indicated G with a T. MyoD_Q6 binding sites occur rather frequently at 0.96 matches per 1000 base pairs of random genomic sequence in vertebrates.

MyoD is a tissue-specific bHLH transcription factor which heterodimerizes with E47; the heterodimer binds to the sequence which here contains G474, called an “E-box.”The effect of disrupting this putative MyoD binding site in the TGF-β1 promoter is unknown.

e. There is also disruption of several potential AP4 (activator protein 4) binding sites, as follows:

(i) An AP4_Q6 binding site whose 3′ terminus ends at nucleotide 467 on the (−) strand, and consists of the sequence complementary to 5′-NCCAGCTGWG-3′ (SEQ ID NO: 7). This SNP replaces the indicated G with a T. AP4_Q6 binding sites occur somewhat infrequently with 0.50 matches per 1000 base pairs of random genomic sequence in vertebrates. AP4 is a transcriptional activator, thus disruption of this site is expected to reduce the rate of transcription of the TGF-β1 gene.

(ii) An AP4_Q5 binding site whose 3′ terminus ends at nucleotide 467 on the (−) strand, and consists of the sequence complementary to 5′-NNCAGCTGNN-3′ (SEQ ID NO: 8). This SNP replaces the indicated G with a T. AP4_Q5 binding sites occur somewhat more frequently at 0.96 matches per 1000 base pairs of random genomic sequence in vertebrates. AP4 is a transcriptional activator, thus disruption of this site is expected to reduce the rate of transcription of the TGF-β 1 gene.

From the standpoint of molecular epidemiology, the G474-->T SNP appears to be important for hypertension. Association of this SNP with essential hypertension suggests an entirely novel mechanism for the disease.

### Example 3

### C to G Substitution at Position 510 of Human TGF-β1 Promoter

TABLE 3
C to G Substitution at Position 510 of Human TGF-β1 Promoter
ALLELE FREQUENCIES

| | C | G |
|---|---|---|
| CONTROL (n = 56 chromosomes): | 51 | 5 |
| Caucasian men | 91% | 9% |
| DISEASE | | |
| HYPERTENSION (n = 66 chromosomes): | 66 | 0 |
| Caucasian men | 100% | 0% |
| ESRD due to HTN (n = 34 chromosomes): | 34 | 0 |
| Caucasian men | 100% | 0% |

TABLE 4
GENOTYPE FREQUENCIES

| | C/C | C/G | G/G |
|---|---|---|---|
| CONTROL (n = 28 individuals): | 23 | 5 | 0 |
| Caucasian men | 82% | 18% | 0% |
| DISEASE | | | |
| HYPERTENSION (n = 33 individuals): | 33 | 0 | 0 |
| Caucasian men | 100% | 0% | 0% |
| ESRD due to HTN (n = 17 individuals): | 17 | 0 | 0 |
| Caucasian men | 100% | 0% | 0% |

PCR and sequencing were conducted as in Example 1. The PCR primers used were the same as those in Example 2.

The G allele, i.e. the SNP at this position, appears to be protective against essential hypertension, since its frequency is 9% in controls but 0% in white men with hypertension. White men with ESRD due to hypertension similarly lack the G allele, suggesting that it is neutral for the development of ESRD. The genotype frequencies are in agreement so that the frequency of the C/G genotype decreases from 18% in controls to 0% in white male patients with hypertension or ESRD due to hypertension.

These data satisfy Hardy-Weinberg equilibrium for the control sample. A frequency of 0.91 for the C allele (“p”) and 0.09 for the G allele (“q”) among control individuals predicts genotype frequencies of 83% C/C, 17% C/G, and 0% G/G at Hardy-Weinberg equilibrium (p²+2pq+q²=1). The observed genotype frequencies were 82% C/C, 18% C/G, and 0% G/G, in excellent agreement with those predicted for Hardy-Weinberg equilibrium. In contrast, the two disease categories diverge greatly from Hardy-Weinberg equilibrium, consistent with the hypothesis that this SNP is truly disease-associated.

The C510-->G SNP is predicted to disrupt a potential RFX1_{—}01 (X-box binding protein RFX1) binding site beginning at nucleotide 504 on the (+) strand. The binding site consists of the sequence 5′-NNGTNRCNNRGYAACNN-3′ (SEQ ID NO: 9). This SNP replaces the indicated C with a G. RFX1_{—}01 sites occur relatively frequently with 0.94 matches per 1000 base pairs of random genomic sequence in vertebrates.

RFX1 is a potent transcriptional repressor (Katan-Khaykovich et al., J Mol Biol 294:121-137, 1999). Disruption of its binding site in the TGF-β1 promoter is expected to result in a lower rate of TGF-β1 transcription, and a lower rate of TGF-β1 signaling, as discussed above. The C510-->G SNP is therefore expected to be protective for any process dependent on increased TGF-β1 signaling.

It is interesting that patients with hypertension but no renal failure have the same frequency of the protective G allele as patients with ESRD due to hypertension. This suggests that hypertension itself may be due to increased TGF-β1 signaling. Such a mechanism would be novel.

From the standpoint of molecular epidemiology, the C510-->G SNP appears to protect against hypertension. Involvement of this SNP suggests that increased TGF-β1 signaling may be associated with essential hypertension.

### Example 4

TABLE 5
G to T Substitution at Position 546 of Human TGF-β1 Promoter
ALLELE FREQUENCIES

| | G | A |
|---|---|---|
| CONTROL (n = 54 chromosomes): | 45 | 9 |
| Caucasian men | 83% | 17% |
| DISEASE | | |
| HYPERTENSION (n = 66 chromosomes): | 64 | 2 |
| Caucasian men | 97% | 3% |
| ESRD due to HTN (n = 34 chromosomes): | 34 | 0 |
| Caucasian men | 100% | 0% |

TABLE 6
GENOTYPE FREQUENCIES

| | G/G | G/A | A/A |
|---|---|---|---|
| CONTROL (n = 27 individuals): | 18 | 9 | 0 |
| Caucasian men | 67% | 33% | 0% |
| DISEASE | | | |
| HYPERTENSION (n = 33 individuals): | 32 | 0 | 1 |
| Caucasian men | 97% | 0% | 3% |
| ESRD due to HTN (n = 17 individuals): | 17 | 0 | 0 |
| Caucasian men | 100% | 0% | 0% |

PCR and sequencing were conducted as in Example 1. The PCR primers used were the same as those in Example 2.

The frequency of the reference G allele is just as high (100%) among white men with ESRD due to hypertension as among white men with hypertension (97%). Both are considerably higher than the G allele frequency in a control sample of white men (83%). The genotype frequencies are equally dramatic. The frequency of the G/G genotype increases markedly from control (67%) to hypertension (97%). The frequency of the G/G genotype in ESRD with hypertension (100%) is essentially the same as in the hypertension group (97%).

These data satisfy Hardy-Weinberg equilibrium for the control sample, given the sample size. A frequency of 0.83 for the G allele (“p”) and 0.17 for the A allele (“q”) among control individuals predicts genotype frequencies of 69% GIG, 28% G/A, and 3% A/A at Hardy-Weinberg equilibrium (p²+2pq+q²=1). The observed genotype frequencies were 67% G/G, 33% G/A, and 0% A/A, in reasonable agreement with those predicted for Hardy-Weinberg equilibrium. Both essential hypertension and ESRD due to hypertension diverge greatly from Hardy-Weinberg equilibrium, consistent with the hypothesis that this SNP is associated with disease.

The G546-->A SNP is predicted to disrupt a single IK2 (Ikaros 2) binding site beginning at nucleotide 542 on the (+) strand of the TGF-β1 promoter. The binding site consists of the sequence 5′-NNNYGGGAWNNN-3′ (SEQ ID NO: 10). This SNP replaces the indicated G with an A. IK2 binding sites occur relatively frequently with 3.95 matches per 1000 base pairs of random genomic sequence in vertebrates.

IK2 is a transcriptional activator (Croager et al., J. Interferon Cytokine Res. 18:915-920, 1998), so disruption of its binding site in the TGF-β1 promoter is expected to result in a lower rate of TGF-β1 transcription, and a lower rate of TGF-β1 signaling, as discussed above. The G546-->A SNP is therefore expected to be protective for the development of renal failure, since the currently accepted model of progression of chronic renal failure involves increased TGF-β1 signaling. These data are in agreement with such a model. Among patients with end-stage renal disease, the G/G genotype (100%) is present more often than in the control population (67%).

It is surprising that essential hypertension has the same G/G genotype frequency (97%) as ESRD due to hypertension (100%). Thus, preservation of the IK2 binding site in the TGF-β1 promoter appears to be important for the development of hypertension. The unexpected association of increased TGF-β1 transcription with hypertension was also seen with the C510-->G SNP.

From the standpoint of molecular epidemiology the G546-->A SNP appears to be associated strongly with hypertension. These data indicate that the reference sequence “G” allele contributes significantly towards hypertension. Put differently, the A allele, i.e. the single nucleotide polymorphism at this position, appears to be strongly protective against hypertension. This association suggests a novel mechanism for essential hypertension, namely increased TGF-β1 signaling.

### Example 5

TABLE 7
G to A Substitution at Position 563 of Human TGF-β1 Promoter
ALLELE FREQUENCIES FOR CAUCASIAN MEN

| | G | A |
|---|---|---|
| CONTROL (n = 50 chromosomes): | 44 | 6 |
| Caucasian men | 88% | 12% |
| DISEASE | | |
| HYPERTENSION (n = 62 chromosomes) | 55 | 7 |
| Caucasian men | 89% | 12% |

TABLE 8
ALLELE FREQUENCY FOR AFRICAN-AMERICAN
MEN AND WOMEN

| | G | % | A | % |
|---|---|---|---|---|
| CONTROLS (n = 248 chromosomes) | 240 | 97% | 8 | 3.2% |
| DISEASE | | | | |
| HYPERTENSION (n = 180 chromosomes) | 162 | 90% | 18 | 10% |

TABLE 9
GENOTYPE FREQUENCIES FOR CAUCASIAN MEN

| | G/G | G/A | A/A |
|---|---|---|---|
| CONTROL (n = 25 individuals): | 19 | 6 | 0 |
| Caucasian men | 76% | 24% | 0% |
| DISEASE | | | |
| HYPERTENSION (n = 31 individuals): | 25 | 5 | 1 |
| Caucasian men | 81% | 17% | 3% |

TABLE 10
GENOTYPE FREQUENCIES FOR AFRICAN AMERICAN
MEN AND WOMEN

| | G/G | G/A | A/A |
|---|---|---|---|
| CONTROLS (n = 124 individuals) | 116 | 8 | 0 |
| | 93.5% | 6.5% | 0.0% |
| DISEASE | | | |
| HYPERTENSION (n = 90 individuals) | 72 | 18 | 0 |
| | 80.0% | 20.0% | 0.0% |

Allele-Specific Odds Ratios Three basic statistics were calculated during this analysis: a point estimate, 95% confidence interval, and a likelihood (p-value). A simple odds ratio is used as the point estimate of association. The 95% confidence intervals were calculated using the asymptotic method. P-values for differences in allele or genotype frequencies between cases and controls were calculated using Pearson and Likelihood Ratio chi-squares, evaluated with a two-sided alternative to the null hypothesis of no association. All calculations were done using the SAS suite of statistical software, version 8.1 (SAS Institute, Cary, N.C.).

For the data related to African-American men and women, the susceptibility allele is indicated below, as well as the odds ratio (OR). The allele which is present more often in the given disease category was chosen as the susceptibility allele. Haldane's correction was used when the denominator was zero, and is so indicated with an “H”. If the odds ratio (OR) is >1.5, the 95% confidence interval (C.I.) is also given. An odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios<1.5).” Id. at 66.

An example of the odds ratio calculation is given below:

Hypertension:

| | Cases | Controls |
|---|---|---|
| A | 18 | 8 |
| G | 162 | 240 |

The odds ratio that the A allele is the susceptibility allele for African Americans with hypertension is (18)(240)/(162)(8)=3.3. Odds ratios of 1.5 or greater are highlighted below.

TABLE 11
ALLELE-SPECIFIC ODDS RATIOS

| | SUSCEPTIBILITY | | | |
|---|---|---|---|---|
| DISEASE | ALLELE | OR | 95% C.I. | P Value |
| HYPERTENSION | A | 3.3 | 1.4-7.8 | 0.007 |

Genotype-Specific Odds Ratios

The susceptibility allele (S) is indicated; the alternative allele at this locus is defined as the protective allele (P). Also presented is the odds ratio (OR) for the SS and SP genotypes; the odds ratio for the PP genotype is 1, since it is the reference group, and is not presented separately. For odds ratios>1.5, the 95% confidence interval (C.I.) is also given in parentheses. An odds ratio of 1.5 was chosen as the threshold of significance based on the recommendation of Austin et al. in Epidemiol. Rev. 16:65-76, 1994. “[E]pidemiology in general and case-control studies in particular are not well suited for detecting weak associations (odds ratios<1.5).” Id. at 66.

An example is worked below, assuming that A is the susceptibility allele (S), and G is the protective allele (P).

Hypertension:

| | Cases | Controls |
|---|---|---|
| AA (SS) | 0 | 0 |
| AG (SP) | 18 | 8 |
| GG (PP) | 72 | 116 |

Applying Haldane's correction because the denominator contains a 0, the above 2×3 table becomes:

Hypertension

| | Cases | Controls | Odds Ratio |
|---|---|---|---|
| AA (SS) | 1 | 1 | (1)(233)/(1)(145) = 1.6 |
| AG (SP) | 37 | 17 | (37)(233)/(17)(145) = 3.6 |
| GG (PP) | 145 | 233 | 1.0 (by definition) |

Where Haldane's zero cell correction was used, the odds ratio is so indicated with a superscript “H”. The odds ratios for individual genotypes are given below.

To minimize confusion, genotype-specific odds ratios are presented only for diseases in which the allele-specific odds ratio was at least 1.5. Genotype-specific odds ratios of 1.5 or more are highlighted.

TABLE 12

| | RISK | | | | 95% | |
|---|---|---|---|---|---|---|
| DISEASE | ALLELE | SS O.R. | 95% C.I. | SP O.R. | C.I. | p-value |
| HYPER- | A | 1.6^{H} | 0.1-26.2 | 3.6 | 1.5-8.8 | 0.002 |
| TENSION | | | | | | |

PCR and sequencing were conducted as in Example 1. The PCR primers used were the same as those in Example 2.

Hardy-Weinberg analysis was conducted on both case and control samples for each population group.

Results

Caucasian Men

Although the allele frequencies are similar among the control and disease groups (frequency of the reference G allele is 88% among white male controls, 89% among white male hypertensives, there is a marked difference in the genotype frequencies. The G/G genotype frequency increases from control (76%) to hypertensive patients (81%). The non-G/G genotypes, G/A and A/A taken together, decrease from 24% among the control group to 20% among white male hypertensives. These data suggest that the G/G genotype is a moderate risk factor for hypertension.

These data satisfy Hardy-Weinberg equilibrium for the control sample, considering the sample size. A frequency of 0.88 for the G allele (“p”) and 0.12 for the A allele (“q”) among control individuals predicts genotype frequencies of 77% G/G, 21% G/A, and 1% A/A at Hardy-Weinberg equilibrium (p²+2pq+q²=1). The observed genotype frequencies were 76% C/C, 24% C/G, and 0% G/G, in very close agreement with those predicted for Hardy-Weinberg equilibrium. For white males, hypertension diverges from Hardy-Weinberg equilibrium, consistent with the hypothesis that this SNP is associated with hypertension.

African-American Men and Women

A frequency of 0.968 for the G allele (“q”) and 0.032 for the A allele (“p”) among control individuals predicts genotype frequencies of 94.0% G/G, 6.0% G/A, and 0.0% A/A at Hardy-Weinberg equilibrium (p²+2pq+q²=1). The observed genotype frequencies were 96.8% G/G, 20.0% G/T, and 0.0% T/T, in moderate agreement with those predicted for Hardy-Weinberg equilibrium. The chi-square statistic for a test of disequilibrium was 0.025, which has a p-value of 0.87 on 2 degrees of freedom. Thus, the observed genotype frequencies do not deviate significantly from Hardy-Weinberg equilibrium.

A frequency of 0.90 for the G allele (“q”) and 0.10 for the A allele (“p”) among patients with hypertension only predicts genotype frequencies of 81.0% GIG, 18.0% G/A, and 1.0% A/A at Hardy-Weinberg equilibrium (p²+2pq+q²=1). The observed genotype frequencies were 80.0% GIG, 20.0% G/T, and 0.0% T/T, in moderate agreement with those predicted for Hardy-Weinberg equilibrium. The chi-square statistic for a test of disequilibrium was 1.6, which has a p-value of 0.44 on 2 degrees of freedom. Thus, the observed genotype frequencies do not deviate significantly from Hardy-Weinberg equilibrium.

For patients with hypertension only the odds ratio for the A allele was 3.3 [(95% CI, 1.4-7.8), p=0.007]. The odds ratio for the homozygote (A/A) was 1.6 (95% CI, 0.1-26.2), while the odds ratio for the heterozygote (G/A) was 3.6 (95% CI, 1.5-8.8) [p=0.002 for both]. These data suggest that the A allele acts in a co-dominant manner in this patient population. These data further suggest that the TGF-β1 gene is significantly associated with hypertension, i.e. abnormal activity of the TGF-β1 gene predisposes individuals to hypertension.

Analysis

The G563-->A SNP is predicted to disrupt the core sequence of a number of potential transcriptional activators. The G563-->A allele would therefore be expected to be protective for any disease process that involved increased TGF-β1 signaling, such as hypertension. Our observation that the reference allele (G/G genotype) is associated with hypertension suggests a novel mechanism for hypertension. The potential binding sites affected by the G-to-A transition at this position are as follows:

a. The substitution disrupts a potential ATF (activating transcription factor) site, which consists of the complement of 5′-GRNNNACGTCASNG-3′ (SEQ ID NO: 11), whose 3′ terminus ends at nucleotide #556 on the (−) strand. ATF sites occur relatively rarely at 0.34 times per 1000 base pairs of random genomic sequence in vertebrates. Disruption of this site is expected to result in decreased transcription of TGF-β1, leading to an expected decrease in the levels of TGF-β1 mRNA and protein in tissues.

b. The substitution disrupts a potential CREB (cAMP-responsive element binding protein) site. Five variations of this site, all centered at this SNP and requiring G563 for maximal activity, exist. In all of them, this SNP replaces the indicated G by an A, as follows:

(1) CREBP 1_Q2, whose consensus binding sequence is the complement of 5′-NSTKACGTCASN-3′ (SEQ ID NO: 12), has its 3′ terminus at nucleotide #557 on the (−) strand. This sequence occurs only 0.09 times per 1000 base pairs of random genomic sequence in vertebrates, so its disruption by this SNP appears highly significant.

(2) CREB_Q2, whose consensus binding sequence is the complement of 5′-NNTTACKGTCASN-3′ (SEQ ID NO: 13), has its 3′ terminus at nucleotide #557 on the (−) strand. This sequence occurs 0.34 times per 1000 base pairs of random genomic sequence in vertebrates, which is also relatively rare.

(3) CREB_Q4, whose consensus binding sequence is the complement of 5′-NNTKACGTCASN-3′ (SEQ ID NO: 14 has its 3′ terminus at nucleotide #557 on the (−) strand. This sequence occurs 0.34 times per 1000 base pairs of random genomic sequence in vertebrates, which is also relatively rare.

(4) CREB_{—}02, whose consensus binding sequence is the complement of 5′-NNRCGTCANCNN-3′ (SEQ ID NO: 15), has its 3′ terminus at nucleotide #559 on the (−) strand. This sequence occurs less rarely, at 1.12 times per 1000 base pairs of random genomic sequence in vertebrates.

(5) CREB_{—}01, whose consensus binding sequence is the complement of 5′-TKACGTCA-3′, has its 3′ terminus at nucleotide #559 on the (−) strand. This sequence occurs 0.40 times per 1000 base pairs of random genomic sequence in vertebrates, which is relatively rare.

c. The substitution disrupts a potential CREBP 1 CJUN (cAMP-responsive element binding protein/c-Jun heterodimer) binding site, consisting of the complement of 5′-TRACGTCA-3′, whose 3′ terminus ends at nucleotide #559 on the (−) strand. This sequence occurs relatively rarely in vertebrates at 0.22 times per 1000 base pairs of random genomic sequence.

d. Finally, the G563-->A SNP disrupts an API (activator protein 1) site, whose consensus sequence consists of the complement of 5′-WNKNAGTCASY-3′ (SEQ ID NO: 16), whose 3′ terminus ends at nucleotide 558 on the (−) strand. The indicated G is replaced with an A.

This site occurs relatively frequently at 1.82 times per 1000 bases of random genomic sequence in vertebrates.

These data suggest that the reference sequence G563 allele, especially in the homozygous state (G/G genotype), contributes to hypertension. Put differently, the A allele, i.e. the single nucleotide polymorphism at this position, appears to be moderately protective against hypertension.

Other examples of genotype-specific disease associations exist, such as the deletion/deletion (D/D) genotype of the angiotensin 1-converting enzyme (Cambien et al., Nature 359:641-644, 1992). In the case of the ACE insertion/deletion polymorphism, studies often show that the D/D genotype is associated with disease, rather than the D allele. Presumably, only the homozygote (an individual with the GIG genotype, in the case of the G563-->A SNP) exceeds a critical threshold of TGF-β1 signaling. The G/A heterozygote behaves functionally the same as the A/A homozygote, suggesting that compensatory mechanisms may be responsible for the lack of association of either of these genotypes with disease. The nature of any such compensatory mechanisms is unknown.

Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only are possible explanations.

It is to be further understood that the specific embodiments of the present invention as set forth are not intended as being exhaustive or limiting of the invention, and that many alternatives, modifications, and variations will be apparent to those of ordinary skill in the are in light of the foregoing examples and detailed description. Accordingly, this invention is intended to embrace all such alternatives, modifications, and variations that fall within

TABLE 13

| Gene | Region | Location | Wild Type | Variant | SEQ ID |
|---|---|---|---|---|---|
| TGF-β1 | Promoter | 474 | G | T | 1 |
| | | 510 | C | G | 1 |
| | | 546 | G | A | 1 |
| | | 563 | G | A | 1 |

TABLE 14

| Gene | Region | Location | Wild Type | Variant | SEQ ID |
|---|---|---|---|---|---|
| TGF-β1 | Promoter | 474 | G | T | 1 |
| | | 510 | C | G | 1 |
| | | 546 | G | A | 1 |

What is claimed is:

1. A method for diagnosing a genetic susceptibility for a disease, condition, or disorder in a subject comprising:
obtaining a biological sample containing nucleic acid from said subject; and
analyzing said nucleic acid to detect the presence or absence of a single nucleotide polymorphism in the TGF-β1 gene, wherein said single nucleotide polymorphism is associated with a genetic susceptibility for hypertension.

2. The method of claim 1, wherein the TGF-βI gene comprises SEQ ID NO: 1.

3. The method of claim 1, wherein said nucleic acid is DNA, RNA, cDNA or mRNA.

4. The method of claim 2, wherein said single nucleotide polymorphism is located at position 474, 510, 546, or 563 of SEQ ID NO: 1.

5. The method of claim 4, wherein said single nucleotide polymorphism is a selected from the group consisting of G474->T, C474->A, C510->G, G510->C, G546->A, C546->T, G563->A, and C563->T.

6. The method of claim 1, wherein said analysis is accomplished by sequencing, mini sequencing, hybridization, restriction fragment analysis, oligonucleotide ligation assay or allele specific PCR.

7. An isolated polynucleotide comprising at least 10 contiguous nucleotides of SEQ ID NO: 1, or the complements thereof, and containing at least one single nucleotide polymorphism at position 474, 510, 546, or 563 of SEQ ID NO: 1 wherein said at least one single nucleotide polymorphism is associated with hypertension.

8. The isolated polynucleotide of claim 7, wherein at least one single nucleotide polymorphism is selected from the group consisting of G474->T, C474->A, C510->G, G510->C, G546->A, C546->T, G563->A, and C563->T.

9. The isolated polynucleotide of claim 7, wherein said at least one single nucleotide polymorphism is located at the 3′ end of said nucleic acid sequence.

10. The isolated polynucleotide of claim 7, further comprising a detectable label.

11. The isolated nucleic acid sequence of claim 10, wherein said detectable label is selected from the group consisting of radionuclides, fluorophores or fluorochromes, peptides, enzymes, antigens, antibodies, vitamins or steroids.

12. A kit comprising at least one isolated polynucleotide of at least 10 contiguous nucleotides of SEQ ID NO: 1 or the complement thereof, and containing at least one single nucleotide polymorphism associated with hypertension; and instructions for using said polynucleotide for detecting the presence or absence of said at least one single nucleotide polymorphism in said nucleic acid.

13. The kit of claim 12 wherein said at least one single nucleotide polymorphism is located at position 474, 510, 546, or 563 of SEQ ID NO: 1.

14. The kit of claim 13 wherein said at least one single nucleotide polymorphism is selected from the group consisting of G474->T, C474->A, C510->G, G510->C, G546->A, C546->T, G563->A, and C563->T.

15. The kit of claim 12, wherein said single nucleotide polymorphism is located at the 3′ end of said polynucleotide.

16. The kit of claim 12, wherein said polynucleotide further comprises at least one detectable label.

17. The kit of claim 16, wherein said label is chosen from the group consisting of radionuclides, fluorophores or fluorochromes, peptides enzymes, antigens, antibodies, vitamins or steroids.

18. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with hypertension; and instructions for using said polynucleotide for detecting the presence or absence of said single nucleotide polymorphism in a biological sample containing nucleic acid.

19. The kit of claim 18, wherein said at least one polynucleotide further comprises a detectable label.

20. The kit of claim 19, wherein said detectable label is chosen from the group consisting of radionuclides, fluorophores or fluorochromes, peptides, enzymes, antigens, antibodies, vitamins or steroids.

21. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with hypertension; and
treating said subject for said disease, condition or disorder.

22. The method of claim 21 wherein said nucleic acid is selected from the group consisting of DNA, cDNA, RNA and mRNA.

23. The method of claim 21, wherein said at least one single nucleotide polymorphism is located at position 474, 510, 546, or 563 of SEQ ID NO: 1.

24. The method of claim 21 wherein said at least one single nucleotide polymorphism is selected from the group consisting of G474->T, C474->A, C510->G, G510->C, G546->A, C546->T, G563->A, and C563->T.

25. The method of claim 21 wherein said treatment counteracts the effect of said at least one single nucleotide polymorphism detected.

26. A method for diagnosing a genetic susceptibility for a disease, condition, or disorder in a subject comprising:
obtaining a biological sample containing nucleic acid from said subject; and
analyzing said nucleic acid to detect the presence or absence of a single nucleotide polymorphism in the TGF-βI gene, wherein said single nucleotide polymorphism is associated with a genetic susceptability for end stage renal disease due to hypertension.

27. The method of claim 26, wherein the TGF-PI gene comprises SEQ ID NO: 1.

28. The method of claim 26, wherein said nucleic acid is DNA, RNA, cDNA or mRNA.

29. The method of claim 27, wherein said single nucleotide polymorphism is located at position 474, 510, or 546 of SEQ ID NO: 1.

30. The method of claim 29, wherein said single nucleotide polymorphism is a selected from the group consisting of G474->T, C474->A, C510->G, G510->C, G546->A, and C546->T.

31. The method of claim 26, wherein said analysis is accomplished by sequencing, mini sequencing, hybridization, restriction fragment analysis, oligonucleotide ligation assay or allele specific PCR.

32. An isolated polynucleotide comprising at least 10 contiguous nucleotides of SEQ ID NO: 1, or the complements thereof, and containing at least one single nucleotide polymorphism at position 474, 510, or 546 of SEQ ID NO: 1 wherein said at least one single nucleotide polymorphism is associated with end stage renal disease due to hypertension.

33. The isolated polynucleotide of claim 32, wherein at least one single nucleotide polymorphism is selected from the group consisting of G474->T, C474->A, C510->G, G510->C, G546->A, and C546->T.

34. The isolated polynucleotide of claim 32, wherein said at least one single nucleotide polymorphism is located at the 3′ end of said nucleic acid sequence.

35. The isolated polynucleotide of claim 32, further comprising a detectable label.

36. The isolated nucleic acid sequence of claim 34, wherein said detectable label is selected from the group consisting of radionuclides, fluorophores or fluorochromes, peptides, enzymes, antigens, antibodies, vitamins or steroids.

37. A kit comprising at least one isolated polynucleotide of at least 10 contiguous nucleotides of SEQ ID NO: 1 or the complement thereof, and containing at least one single nucleotide polymorphism associated with end stage renal disease due to hypertension; and instructions for using said polynucleotide for detecting the presence or absence of said at least one single nucleotide polymorphism in said nucleic acid.

38. The kit of claim 37 wherein said at least one single nucleotide polymorphism is located at position 474, 510, or 546 of SEQ ID NO: 1.

39. The kit of claim 38 wherein said at least one single nucleotide polymorphism is selected from the group consisting of G474->T, C474->A, C510->G, G510->C, G546->A, and C546->T.

40. The kit of claim 37, wherein said single nucleotide polymorphism is located at the 3′ end of said polynucleotide.

41. The kit of claim 37, wherein said polynucleotide further comprises at least one detectable label.

42. The kit of claim 41, wherein said label is chosen from the group consisting of radionuclides, fluorophores or fluorochromes, peptides enzymes, antigens, antibodies, vitamins or steroids.

43. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with end stage renal disease due to hypertension; and instructions for using said polynucleotide for detecting the presence or absence of said single nucleotide polymorphism in a biological sample containing nucleic acid.

44. The kit of claim 43, wherein said at least one polynucleotide further comprises a detectable label.

45. The kit of claim 44, wherein said detectable label is chosen from the group consisting of radionuclides, fluorophores or fluorochromes, peptides, enzymes, antigens, antibodies, vitamins or steroids.

46. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with end stage renal disease due to hypertension; and
treating said subject for said disease, condition or disorder.

47. The method of claim 46 wherein said nucleic acid is selected from the group consisting of DNA, cDNA, RNA and mRNA.

48. The method of claim 46, wherein said at least one single nucleotide polymorphism is located at position 474, 510, or 546 of SEQ ID NO: 1.

49. The method of claim 46 wherein said at least one single nucleotide polymorphism is selected from the group consisting of G474->T, C474->A, C510->G, G510->C, G546->A, and C546->T.

50. The method of claim 46 wherein said treatment counteracts the effect of said at least one single nucleotide polymorphism detected.


SEQUENCE LISTING (16 sequences)

SEQ ID NO: 1 - DNA, 2205 bp, Homo sapiens
  protein_bind (122)..(131): Putative
ggatccttagcaggggagtaacatggatttggaaagatcactttggctgctgtgtggggatagataagacggtgggagcctagaaaggaggctgggttggaaactctgggacagaaacccagagaggaaaagactgggcctggggtctccagtgagtatcagggagtggggaatcagcaggagtctggtccccacccatccctcctttcccctctctctcctttcctgcaggctggccccggctccatttccaggtgtggtcccaggacagctttggccgctgccagcttgcaggctatggattttgccatgtgcccagtagcccgggcacccaccagctggcctgccccacgtggcggcccctgggcagttggcgagaacagttggcacgggctttcgtgggtggtgggccgcagctgctgcatggggacaccatctacagtggggccgaccgctatcgcctgcacacagctgctggtggcaccgtgcacctggagatcggcctgctgctccgcaacttcgaccgctacggcgtggagtgctgagggactctgcctccaacgtcaccaccatccacaccccggacacccagtgatgggggaggatggcacagtggtcaagagcacagactctagagactgtcagagctgaccccagctaaggcatggcaccgcttctgtcctttctaggacctcggggtccctctgggcccagtttccctatctgtaaattggggacagtaaatgtatggggtcgcagggtgttgagtgacaggaggctgcttagccacatgggaggtgctcagtaaaggagagcaattcttacaggtgtctgcctcctgacccttccatccctcaggtgtcctgttgccccctcctcccactgacaccctccggaggcccccatgttgacagaccctccttctcctaccttgtttcccagcctgactctccttccgttctgggtccccctcctctggtcggctcccctgtgtctcatcccccggattaagccttctccgcctggtcctctttctctggtgacccacaccgcccgcaaagccacagcgcatctggatcacccgctttggtggcgcttggccgccaggaggcagcaccctgtttgcggggcggagccggggagcccgccccctttcccccagggctgaagggacccccctcggagcccgcccacgcgagatgaggacggtggcccagcccccccatgccctccccctgggggccgcccccgctcccgccccgtgcgcttcctgggtggggccgggggcggcttcaaaaccccctgccgacccagccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatg

SEQ ID NO: 2 - DNA, 22 bp, Artificial Sequence
  Primer
tgcatggggacaccatctacag

SEQ ID NO: 3 - DNA, 22 bp, Artificial Sequence
  Primer
tcttgaccactgtgccatcctc

SEQ ID NO: 4 - DNA, 16 bp, Homo sapiens
  primer_bind (1)..(16)
nnkaacacctgyknnn

SEQ ID NO: 5 - DNA, 13 bp, Homo sapiens
  primer_bind (1)..(13)
nnncacctgcnns

SEQ ID NO: 6 - DNA, 10 bp, Homo sapiens
  primer_bind (1)..(10)
rncagntgnn

SEQ ID NO: 7 - DNA, 10 bp, Homo sapiens
  primer_bind (1)..(10)
nccagctgwg

SEQ ID NO: 8 - DNA, 10 bp, Homo sapiens
  primer_bind (1)..(10)
nncagctgnn

SEQ ID NO: 9 - DNA, 17 bp, Homo sapiens
  primer_bind (1)..(17)
nngtnrcnnrgyaacnn

SEQ ID NO: 10 - DNA, 12 bp, Homo sapiens
  primer_bind (1)..(12)
nnnygggawnnn

SEQ ID NO: 11 - DNA, 14 bp, Homo sapiens
  primer_bind (1)..(14)
grnnnacgtcasng

SEQ ID NO: 12 - DNA, 12 bp, Homo sapiens
  primer_bind (1)..(12)
nstkacgtcasn

SEQ ID NO: 13 - DNA, 13 bp, Homo sapiens
  primer_bind (1)..(13)
nnttackgtcasn

SEQ ID NO: 14 - DNA, 12 bp, Homo sapiens
  primer_bind (1)..(12)
nntkacgtcasn

SEQ ID NO: 15 - DNA, 12 bp, Homo sapiens
  primer_bind (1)..(12)
nnrcgtcancnn

SEQ ID NO: 16 - DNA, 11 bp, Homo sapiens
  primer_bind (1)..(11)
wnknagtcasy